(19) United States
(12) Patent Application Publication
(10) Pub. No.: US 2016/0106131 A1
(43) Pub. Date: Apr. 21, 2016

(54) USE OF HMB TO IMPROVE QUALITY OF LIFE FOR HOSPITALIZED PATIENTS

(71) Applicant: ABBOTT LABORATORIES, Abbott Park, IL (US)

(72) Inventors: Menghua LUO, New Albany, OH (US); Jeffrey NELSON, Dublin, OH (US); Anne VOSS, Columbus, OH (US); Owen KELLY, Pataskala, OH (US); Geraldine BAGGS, Columbus, OH (US); Min TIAN, New Albany, OH (US); Jamie PARTRIDGE, Columbus, OH (US)

(73) Assignee: ABBOTT LABORATORIES, Abbott Park, IL (US)

(21) Appl. No.: 14/982,488

(22) Filed: Dec. 29, 2015

Related U.S. Application Data
(63) Continuation-in-part of application No. 14/406,886, filed on Dec. 10, 2014.
(60) Provisional application No. 61/658,078, filed on Jun. 11, 2012.

(51) Int. Cl. A23L 1/305; A61K 31/19

(52) U.S. Cl. CPC A23L 1/3051; A61K 31/19; A23L 1/296; A23L 1/293; A23V 2002/00

(57) ABSTRACT

A method of care is described that includes administering an effective amount of β-hydroxy-β-methylbutyrate (HMB) to an adult or elderly patient to improve the patient's quality of life during or after hospitalization of the patient. The improved quality of life may encompass the general health, physical health, or mental health of the patient.

### RELATED APPLICATIONS

This application is a continuation-in-part of U.S. patent application Ser. No. 14/406,886, with a filing date of Dec. 10, 2014, which was the U.S. national phase entry of PCT/US2013/044899, with an international filing date of Jun. 10, 2013, which claims priority to and the benefit of U.S. Provisional Patent Application Ser. No. 61/658,078, entitled “USE OF HMB TO IMPROVE HEALTH OUTCOMES FOR HOSPITALIZED PATIENTS” and filed Jun. 11, 2012, all of which are incorporated herein by reference in their entirety.

### BACKGROUND

The elderly (65 years of age or older) account for 38% of hospital stays and utilize close to 50% of total hospital expenditures in the US. Acute illness-related short-stay hospitalization in the elderly is frequently associated with a loss of lean body mass (LBM) and function, which can lead to loss of independence, a need for post-discharge assistance, long-term institutionalization, and caregiver burden.

Regardless of the type of acute illness, the prolonged period of immobility or bed rest alone has a pronounced effect on rapid muscle atrophy, which is primarily due to decreased protein synthesis and/or increased protein degradation. With illness or injury, there is a greater extent of LBM loss. In three separate studies, the effect of bed rest on LBM loss was assessed in healthy young volunteers, healthy elderly volunteers, and elderly inpatients. Healthy young volunteers lost 2% (˜500 g=1.1 lb) of LBM in the leg during 28 days of bed rest, healthy elderly subjects lost 10% (˜1000 g=2.2 lb) of leg LBM during 10 days of bed rest, while elderly inpatients lost greater than 10% of leg LBM within only 3 days of hospitalization. Functional impairment occurs with LBM loss, with 65% of elderly patients experience a decline in mobility with hospitalization, and 35% report decreased activities of daily living.

Other deleterious factors associated with hospitalization, such as physiological stress and malnutrition, may result in greater extent of loss in muscle mass and function. It has been estimated that 25-60% of geriatric patients in hospitals and nursing homes have shown evidence of malnutrition. In the acute care hospital setting, around 21% of patients have an average daily in-hospital nutrient intake of less than 50% of their calculated maintenance energy requirements.

These deleterious factors associated with hospitalization (e.g., muscle atrophy, physiological stress, malnutrition) may also contribute to mental health problems such as depression and anxiety. A reduction in physical health and mental health may result in a reduced quality of life for the patient during and after hospitalization.

### SUMMARY

In one aspect, a method of care is provided that includes administering an effective amount of β-hydroxy-β-methylbutyrate (HMB) to an adult patient to improve the patient's quality of life subsequent to hospitalization of the patient. HMB may also be administered to the patient during hospitalization.

In another aspect, a method of care is provided that includes administering from about 1 to 10 g/day ofβ-hydroxy-β-methylbutyrate (HMB) in a liquid nutritional supplement at least once daily to an elderly patient to improve the elderly patient's quality of life up to about 1 year after hospitalization of the patient. HMB may also be administered to the elderly patient during hospitalization.

### DETAILED DESCRIPTION

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

For the purpose of the present disclosure, the following terms are defined:

The term “adult,” as used herein, refers to an individual of at least 21 years of age.

The terms “elderly” and “older,” as used herein, are used interchangeably to refer to an individual or groups of people that are at least 45 years of age, including at least 50 years of age, at least 55 years of age, at least 60 years of age, at least 65 years of age, at least 70 years of age, at least 75 years of age, and including at least 80 years of age or greater.

The term “patient,” as used herein, refers to a male or female human who is being treated for a medical condition.

The term “hospital,” as used herein, is a health care institution providing patient treatment by specialized staff (e.g., physicians, surgeons, nurses, and dieticians) and equipment. Hospitals include general hospitals and specialized hospitals. General hospitals are set up to deal with many kinds of disease and injury, and typically have an emergency department to deal with immediate and urgent threats to health. Specialized hospitals include trauma centers, rehabilitation hospitals, children's hospitals, seniors' (geriatric) hospitals, teaching hospitals, and hospitals for dealing with specific medical needs such as psychiatric problems and certain disease categories such as cardiac, oncology, or orthopedic problems. A hospital includes bedding and other facilities required for inpatient care, as opposed to a health care institution such as a clinic that only provides for outpatient care. Hospitalization refers to the act of a patient being entered or staying within a hospital.

The terms “β-hydroxy-β-methylbutyrate” and “HMB” are used interchangeably herein to refer to the chemical that has the IUPAC name 3-hydroxy-3-methylbutanoic acid. It should be understood that all references to β-hydroxy-β-methylbutyrate and HMB in the present disclosure include HMB as the free acid, and also any salt, anhydrous salt, ester, lactone, or other non-toxic HMB compound that provides a bioavailable form of HMB suitable for administration. Non-limiting examples of suitable HMB compounds for use herein include the hydrated or anhydrous salt of sodium HMB, potassium HMB, calcium HMB, or other non-toxic salts.

The term “formulation” refers to a mixture comprising an active ingredient (e.g., HMB) and other optional ingredients, diluents, excipients, flavors, colorants, stabilizers, etc. The formulation may be administered to a patient to deliver a known quantity of the active ingredient.

The term “nutritional composition” refers to a formulation intended to further provide at least one macronutrient (e.g., protein, carbohydrate, or fat) and other optional ingredients (e.g., vitamins or minerals), for the nourishment of the patient. Nutritional compositions can be a sole source or a supplemental source of nourishment to the patient.

The term “ready to serve” or “RTS” refers to a nutritional composition that is ready to be administered to or consumed by the patient, without further preparation other than opening the packaging. For example, a RTS nutritional beverage may be consumed by the patient without being mixed with, dissolved in, or diluted by a potable liquid (e.g., water, milk, or juice). In another example, a RTS nutritional food may be consumed by the patient without being heated, cooked, baked, mixed with other ingredients, or otherwise prepared prior to consumption.

The term “an effective amount” refers to the amount of an active ingredient which will achieve a stated goal. For the purpose of this disclosure, the active ingredient is β-hydroxy-β-methylbutyrate, and the goal is to improve the quality of life for a patient. The effective amount may be administered in one or more doses.

The term “quality of life” refers to a combination of factors (physical health, mental health, bodily pain, daily activities, social functioning, emotional functioning, etc.) that interact to shape a patient's behavior and attitude towards her or his present and future medical outcome. Standardized methods have been developed to evaluate a patient's view of her or his quality of life when compared with other populations. Two such methods are the EQ-5D Instrument (EuroQol Research Foundation, Rotterdam, NL) and the SF-36 Health Survey (QualityMetric Incorporated, Lincoln, R.I., US).

The term “Subjective Global Assessment” or “SGA” refers to a widely used practical assessment tool of malnutrition. The SGA provides a classification of malnutrition based on both medical history and clinician observations. The SGA is widely used in research settings to document and classify patients into one of three categories of nutrition status. An “A” rating means the patient is well-nourished; a “B” rating means the patient is mildly to moderately malnourished; and a “C” rating means that the patient is severely malnourished.

To the extent that the terms “includes,” “including,” “contains,” or “containing” are used herein, they are intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use.

All percentages, parts, and ratios as used herein are by weight of the total product, unless specified otherwise. All such weights as they pertain to listed ingredients are based on the active ingredients and, therefore, do not include solvents or by-products that may be included in commercially available materials, unless specified otherwise. In addition, the recitations of numerical ranges by endpoints include all numbers subsumed within that range (e.g., 1 to 5 includes 1, 1.5, 2, 2.75, 3, 3.80, 4, 5, etc.).

All references to singular characteristics or limitations of the present disclosure shall include the corresponding plural characteristics or limitations, and vice versa, unless otherwise specified or clearly implied to the contrary by the context in which the reference is made.

A method of care is described that includes administering an effective amount of β-hydroxy-β-methylbutyrate (HMB) to an adult or elderly patient to improve the quality of life for the patient during or subsequent to hospitalization. While HMB will typically provide the most benefit for elderly patients, it can also be helpful for treating adult patients, in particular those whose health has been compromised as a result of disease, treatment side-effects, prolonged bed rest, malnourishment, or mental health problems.

β-hydroxy-β-methylbutyrate (HMB) is a metabolite of the essential amino acid leucine, and has the IUPAC name 3-hydroxy-3-methylbutanoic acid. A preferred form of HMB is the calcium salt of HMB, also designated as Ca-HMB, which is most typically the monohydrate calcium salt. HMB or Ca-HMB used for the disclosed method of care can come from any source. Calcium HMB monohydrate is commercially available from Technical Sourcing International (TSI) of Salt Lake City, Utah. Note that all of the amounts of HMB described herein are based on use of Ca-HMB. Although the calcium monohydrate salt is the preferred form of HMB for use herein, other suitable forms of HMB include HMB as a free acid, a salt, an anhydrous salt, an ester, a lactone, or other chemical forms that provide a bioavailable form of HMB suitable for administration. Non-limiting examples of suitable salts of HMB for use herein include HMB salts, hydrated or anhydrous, of sodium, potassium, calcium, or other non-toxic salt forms.

Malnourishment is a condition frequently seen in hospitalized elderly patients. A patient who is malnourished is a patient whose nutrient intake cannot meet increased demand due to illness or other conditions or a patient who has been receiving an unbalanced diet in which certain nutrients are lacking, in excess (too high an intake), or in the wrong proportions. Malnourishment can be readily identified by those skilled in the art. Patients who are malnourished can be expected to benefit particularly from HMB that is provided in a nutritional composition. Accordingly, in some embodiments, the patient being administered an effective amount of HMB is malnourished prior to administration of HMB.

HMB is incorporated in suitable formulations and then, in accordance with the methods of the invention, administered to a patient in a form adapted to the chosen route of administration. The formulations include, but are not limited to, those suitable for oral or non-oral (including subcutaneous, intramuscular, intraperitoneal, intratumoral, and intravenous) administration. Oral administration, as defined herein, includes any form of administration in which HMB passes through the esophagus of the patient. For example, oral administration includes nasogastric intubation, in which a tube is run from through the nose to the stomach of the patient to administer food, water, or medication.

Formulations for oral administration (“oral formulations”) include any solid, liquid, or powder formulation suitable for use herein, provided that such a formulation allows for the safe and effective oral delivery of HMB and optional nutritive components. In some embodiments, the oral formulation is a liquid nutritional composition. In some embodiments, the oral formulation is a ready to serve (RTS) liquid nutritional composition. In some embodiments, the oral formulation is a food product (e.g., a snack bar, baked good, breakfast cereal, soup, etc.). In some embodiments, the oral formulation may be in the form of medicine-like discrete units such as tablets, troches, capsules, lozenges, wafers, or cachets, each containing a predetermined amount of HMB as a powder or granules, or in a solution or suspension in an aqueous liquid or non-aqueous liquid such as a syrup, an elixir, an emulsion, or a draught.

The concentration of HMB in the formulation may range up to about 10%, including from about 0.01% to about 10%, and also including from about 0.1% to about 5.0% and also including from about 0.5% to about 2%, and also including from about 0.4% to about 1.5% by weight of the formulation. If the HMB formulation is a nutritional composition, the concentration of HMB in the nutritional composition may range up to about 10%, including from about 0.01% to about 10%, and also including from about 0.1% to about 5.0% and also including from about 0.5% to about 2%, and also including from about 0.4% to about 1.5% by weight of the nutritional composition.

A nutritional composition administered to the patients can provide from about 0.1 g/day to about 10 g/day of HMB. Alternately, the nutritional composition can provide about 0.5 g/day to about 10 g/day of HMB, about 1.0 g/day to about 10 g/day, or about 0.5 g/day to about 5 g/day. Patients may be administered one serving per day, two servings per day, three servings per day, or four or more servings per day to receive the desired amount of HMB from the nutritional composition.

HMB can be administered during or subsequent to hospitalization of the patient. For example, HMB can be administered only during the initial hospitalization of the patient, or only during a portion of the initial hospitalization of the patient. Alternately, or in addition, HMB can be administered subsequent to hospitalization of the patient. Administration subsequent to hospitalization includes administration of HMB to the patient beginning immediately or shortly after release from initial hospitalization. For example, administration subsequent to hospitalization can begin up to one week after release, 3 days after release, 1 day after release, or immediately after release. Administration after release can also continue for as long as it is useful for the patient to be receiving HMB. For example, administration can continue for one month, three months, six months, nine months, or a year or more after release from hospitalization.

In a preferred embodiment, HMB is administered to the patient as part of a nutritional composition. The nutritional composition includes one or more ingredients that help satisfy the patients' nutritional requirements, in addition to providing a useful formulation for HMB. For example, the nutritional composition can include a fat, a carbohydrate, or a protein. Preferably, the nutritional composition includes at least one source of fat, at least one source of carbohydrate, and at least one source of protein. In some embodiments, the nutritional composition can be formulated to provide a specialized nutritional product for use in patients afflicted with specific diseases or conditions. Many different sources and types of proteins, lipids, and carbohydrates are known and can be used in nutritional compositions including HMB.

Examples of solid product forms of nutritional compositions that are suitable for use herein include snack and meal replacement products, including those formulated as bars, sticks, cookies, breads, cakes, frozen liquids, candy, breakfast cereals, powders, granulated solids, snack chips or bites, frozen or retorted entrees, and so forth. The nutritional composition can also be in a form that falls between solid and liquid, such as semi-solid or semi-liquid compositions (e.g., ice cream, yogurt, pudding, or gel).

Examples of liquid product forms of nutritional compositions suitable for use herein include snack and meal replacement beverage products, hot or cold beverages, carbonated or non-carbonated beverages, juices or other acidified beverages, milk or soy-based beverages, shakes, coffees, teas, compositions for administration by nasogastric intubation, and so forth. These liquid nutritional compositions are most typical formulated as suspensions or emulsions, but can also be formulated in any other suitable form such as clear liquids, substantially clear liquids, liquid gels, and so forth.

The nutritional compositions comprising HMB include one or more ingredients that help satisfy the patients' nutritional requirements. The optional nutrients can provide up to about 1000 kcal of energy per serving or dose, including from about 25 to about 900 kcal, from about 75 to about 700 kcal, from about 150 to about 500 kcal, from about 350 to about 500 kcal, or from about 200 to about 300 kcal.

Carbohydrates suitable for use in the nutritional compositions may be simple, complex, or variations or combinations thereof. Non-limiting examples of suitable carbohydrates include hydrolyzed or modified starch or cornstarch, maltodextrin, glucose polymers, sucrose, corn syrup, corn syrup solids, rice-derived carbohydrate, glucose, fructose, lactone, high fructose corn syrup, indigestible oligosaccharides (e.g., fructooligosaccharides), soluble or insoluble fiber, honey, sugar alcohols (e.g., maltitol, erythritol, sorbitol), and combinations thereof.

Proteins suitable for use in the nutritional compositions, in addition to HMB compounds described herein, include hydrolyzed, partially hydrolyzed or non-hydrolyzed proteins or protein sources, and can be derived from any known or otherwise suitable source such as milk (e.g., casein or whey), animal (e.g., meat or fish), cereal (e.g., rice or corn), vegetable (e.g., soy or nut), or combinations thereof. In some embodiments, the nutritional composition may include a high amount of protein. For example, the nutritional composition may include at least 10 grams of protein.

Fats suitable for use in the compositions include coconut oil, fractionated coconut oil, soy oil, corn oil, olive oil, safflower oil, high oleic safflower oil, MCT oil (medium chain triglycerides), sunflower oil, high oleic sunflower oil, palm and palm kernel oils, palm olein, canola oil, marine oils, cottonseed oils, and combinations thereof.

HMB-containing nutritional compositions may also include other ingredients that may modify the physical, nutritional, chemical, hedonic, or processing characteristics of the product or serve as pharmaceutical or additional nutritional components. Non-limiting examples of such optional ingredients include preservatives, antioxidants, emulsifying agents, buffers, fructooligosaccharides, chromium picolinate, pharmaceutical additives, colorants, flavors or masking agents, thickening agents and stabilizers, artificial sweeteners, hydrocolloids such as guar gum, xanthan gum, carrageenan, gellan gum, gum acacia, and so forth.

HMB-containing nutritional compositions may also include vitamins or related nutrients, examples of which include vitamin A, vitamin D, vitamin E, vitamin K, thiamine, riboflavin, pyridoxine, vitamin B12, carotenoids, niacin, folic acid, pantothenic acid, biotin, vitamin C, choline, inositol, and salts and combinations thereof.

Examples of preferred nutritional compositions are described in Tables 1A and 1B below, with the specific ingredients provided immediately thereafter.

TABLE 1A

| | | Vanilla or Strawberry | Chocolate Flavored |
|---|---|---|---|
| | | Flavored Nutritional | Nutritional |
| | | Composition | Composition |
| | UNIT | per 8 fl oz | per 8 fl oz |
| Energy EU | kcal | 350 | 350 |
| Protein | g | 20 | 20 |
| Fat | g | 11 | 11 |
| Linoleic acid | g | 3 | 3 |
| Carbohydrate | g | 44 | 45 |
| Fructooligo- | g | 3 | 2 |
| saccharide | | | |
| Sugar | g | 20 | 20 |
| Ca-HMB | g | 1.5 | 1.5 |
| VITAMINS | | | |
| Vitamin A | IU | 1000 | 1000 |
| (Palmitate) | | | |
| Vitamin A | IU | 0 | 0 |
| (B-Carotene) | | | |
| Vitamin D₃ | IU | 160 | 160 |
| Vitamin E | IU | 30 | 30 |
| Vitamin K₁ | mcg | 20 | 20 |
| Vitamin C | mg | 60 | 60 |
| Folic Acid | mcg | 200 | 200 |
| Vitamin B₁ | mg | 0.38 | 0.38 |
| Vitamin B₂ | mg | 0.43 | 0.43 |
| Vitamin B₆ | mg | 0.5 | 0.5 |
| Vitamin B₁₂ | mcg | 3 | 3 |
| Niacin | mg | 5 | 5 |
| Pantothenate | mg | 2.5 | 2.5 |
| Biotin | mcg | 75 | 75 |
| L-carnitine | mg | 43 | 43 |
| Choline | mg | 83 | 83 |
| MINERALS | | | |
| Sodium | mg | 240 | 240 |
| Potassium | mg | 560 | 630 |
| Chloride | mg | 150 | 150 |
| Calcium | mg | 500 | 500 |
| Phosphorus | mg | 350 | 350 |
| Magnesium | mg | 100 | 100 |
| Iron | mg | 4.5 | 4.5 |
| Zinc | mg | 15 | 15 |
| Manganese | mg | 0.50 | 0.70 |
| Copper | mg | 0.50 | 0.75 |
| Iodine | mcg | 25 | 25 |
| Selenium | mcg | 30 | 25 |
| Chromium | mcg | 30 | 40 |
| Molybdenum | mcg | 30 | 30 |

In certain embodiments disclosed in Table 1A, the ingredients of the nutritional composition comprise water, corn syrup, sucrose, milk protein concentrate, sodium caseinate, canola oil, corn oil, fructooligosaccharides, soy protein isolate, calcium HMB, whey protein concentrate, potassium citrate, natural and artificial flavors, potassium phosphate, lecithin, cellulose gel, magnesium hydroxide, calcium carbonate, ascorbic acid, calcium phosphate, choline chloride, sodium chloride, sodium phosphate, potassium hydroxide, zinc sulfate, cellulose gum, 1-carnitine, carrageenan, dl-alpha-tocopherol acetate, dextrose, ferrous sulfate, maltodextrin, niacinamide, gellan gum, calcium pantothenate, citric acid, cupric sulfate, manganese sulfate, chromium chloride, thiamine chloride hydrochloride, coconut oil, vitamin A palmitate pyridoxine hydrochloride, riboflavin, folic acid, biotin, sodium selenate, sodium molybdate, potassium iodide, phylloquinone, cyanocobalamin, and vitamin D3.

TABLE 1B

| | | Vanilla |
|---|---|---|
| | | Nutritional |
| | | Composition |
| | UNIT | per 8 fl oz |
| Energy EU | kcal | 350 |
| Protein | g | 13 |
| Fat | g | 11 |
| Linoleic acid | g | 0.65 |
| Carbohydrate | g | 51 |
| Fructooligo- | g | 3 |
| saccharide | | |
| Sugar | g | 20 |
| Ca-HMB | g | 1.5 |
| VITAMINS | | |
| Vitamin A | IU | 1250 |
| (Palmitate) | | |
| Vitamin A | IU | 0 |
| (B-Carotene) | | |
| Vitamin D₃ | IU | 160 |
| Vitamin E | IU | 9 |
| Vitamin K₁ | mcg | 20 |
| Vitamin C | mg | 36 |
| Folic Acid | mcg | 100 |
| Vitamin B₁ | mg | 0.38 |
| Vitamin B₂ | mg | 0.43 |
| Vitamin B₆ | mg | 0.5 |
| Vitamin B₁₂ | mcg | 1.5 |
| Niacin | mg | 5 |
| Pantothenate | mg | 2.5 |
| Biotin | mcg | 75 |
| L-carnitine | mg | 45 |
| Choline | mg | 83 |
| MINERALS | | |
| Sodium | mg | 240 |
| Potassium | mg | 560 |
| Chloride | mg | 68 |
| Calcium | mg | 350 |
| Phosphorus | mg | 350 |
| Magnesium | mg | 100 |
| Iron | mg | 4.5 |
| Zinc | mg | 3.8 |
| Manganese | mg | 1.2 |
| Copper | mg | 0.50 |
| Iodine | mcg | 38 |
| Selenium | mcg | 21 |
| Chromium | mcg | 30 |
| Molybdenum | mcg | 45 |

In a certain embodiment disclosed in Table 1B, the nutritional composition includes water, corn maltodextrin, sugar, canola oil, sodium caseinate, milk protein concentrate, corn oil, short-chain fructooligosaccharides, soy protein isolate, potassium citrate, calcium HMB, whey protein concentrate, natural and artificial flavors, magnesium phosphate, soy lecithin, sodium phosphate, potassium phosphate, choline chloride, ascorbic acid, calcium carbonate, potassium chloride, 1-carnitine, carrageenan, ferrous sulfate, dl-alpha-tocopherol acetate, zinc sulfate, gellan gum, niacinamide, manganese sulfate, calcium pantothenate, cupric sulfate, vitamin A palmitate, thiamine chloride hydrochloride, pyridoxine hydrochloride, riboflavin, folic acid, chromium chloride, biotin, sodium molybdate, sodium selenate, potassium iodide, phylloquinone, vitamin D3, and cyanocobalamin.

### EXAMPLE

The following example is included for purposes of illustration and is not intended to limit the scope of the invention.

A study on the effects of administering HMB to hospitalized elderly patients during and after hospitalization was performed. The study objectives were to evaluate the effects of elderly patients consuming a high-calorie complete and balanced RTS flavored nutritional beverage providing additional protein and HMB on improved quality of life for these patients.

More specifically, it was the purpose of this study to provide elderly patients with two servings of a high-calorie RTS flavored nutritional beverage containing a total of 700 kcal, 40 g of protein, and 3 g of calcium HMB per day, to examine the self-evaluated general health, physical health, and mental health of the elderly patients, and to study the quality of life for elderly patients both during and after hospitalization.

### Overall Study Design and Plan

This was a randomized, double blinded, controlled, two-group parallel design intervention trial. Elderly eligible patients (≧65 years of age) that were hospitalized as a result of heart failure, acute myocardial infarction, pneumonia, or chronic obstructive pulmonary disease were enrolled within 48 hours of admission. Eligible subjects were stratified based on reason for admission (heart failure, acute myocardial infarction, pneumonia, or chronic obstructive pulmonary disease), gender, and nutritional status, and then randomized into two groups (experimental or control). The control group (n=326) received standard hospital meals, snacks, and two servings per day of a control RTS flavored nutritional beverage. The experimental group (n=326) received standard hospital meals, snacks, and two servings per day of an experimental RTS flavored nutritional beverage containing 1.5 g HMB. The daily volume of study product consumed throughout the study was assessed on Daily Product Intake Forms. Throughout the study, both groups were encouraged to consume the supplement and an adequate dietary intake.

### Selection of Study Population

The study population consisted of older adults admitted to the hospital for heart failure, acute myocardial infarction, chronic obstructive pulmonary disease, or pneumonia. A potential subject was excluded if they exhibit kidney disease, uncontrolled hypertension, liver impairment or failure, diabetes, or any other serious medical illness as diagnosed by the study physician. Subjects were eligible for the study if they met all of the following inclusion criteria:
Subject (male or female) is ≧65 years of age;
Subject recently (within 72 hours) admitted to hospital with a primary diagnosis of heart failure, acute myocardial infarction, pneumonia, or chronic obstructive pulmonary disease;
Subject has a Subjective Global Assessment (SGA) rating of B or C at screening;
Subject has anticipated length of hospital stay ≧3 days and ≦12 days and is expected to consume ≧2 of the 350 kcal servings of study product while in hospital;
Subject is able to consume foods and beverages orally; and
Subject was functionally ambulatory during the 30 days prior to admission.

### Treatments

Upon fulfilling the eligibility criteria and receiving consent of the subject and HIPAA (or other applicable privacy regulation) authorization prior to any study participation, subjects were assigned to treatments using a prospective computer generated randomization plan. Subjects were randomly assigned to the experimental or control study product as listed in Table 2:

TABLE 2
Treatments Administered

| | | | Administration |
|---|---|---|---|
| Study Product | Description | Route | Schedule |
| Control Study | Control RTS Flavored | Oral | Two servings/day |
| Product | Beverage | | |
| Experimental | RTS Flavored Beverage | Oral | Two servings/day |
| Study Product | with HMB | | |

### Identity of Investigational Products

The control study product was a RTS nutritional beverage (Abbott Nutrition, Columbus, Ohio, USA). The control study product provided 48 kcal energy with 12 g carbohydrate and 10 mg Vitamin C. The experimental study product was a chocolate or vanilla flavored RTS nutritional beverage having a formulation corresponding to the respective chocolate and vanilla formulations shown in Table 1A. The experimental study product provided 350 kcal energy with 20 g protein, 11 g fat, and 1.5 g calcium HMB (calcium HMB supplied by Metabolic Technologies, Inc., Ames, Iowa, USA). HMB has a Generally Regarded As Safe (GRAS) status in the U.S.

### Method of Assigning Subjects to Treatment

The randomization was stratified by condition (heart failure, acute myocardial infarction, pneumonia, or chronic obstructive pulmonary disease), gender, and nutritional status by Subjective Global Assessment (B rating=mildly malnourished; C rating=severely malnourished). To maintain best possible balance within each condition, gender, and nutritional status category at a site and within the study overall, a centralized randomization system was used.

### Inpatient Hospital Phase

Study assessments were conducted during the Inpatient Hospital Phase through 90 days Post Hospital Discharge. Following informed consent, major assessments at Screening (Day −1)/Baseline Day (Day 0) included eligibility criteria, medical history, demographics, anthropometrics, severity of illness, and functional and nutritional status. Daily assessments during hospitalization included medications, dietary supplement, and study product intake. At hospital discharge, assessments/procedures included anthropometrics, functional status, medications, dietary supplement, study product intake, and quality of life assessments, using the EQ-5D and SF-36 evaluation instruments.

### Post-Discharge Follow-Up Phase

During the 90-day post-discharge period, the study subjects were asked to continue to supplement their daily intake with two servings per day of the control RTS flavored nutritional beverage or the experimental RTS flavored nutritional beverage containing HMB. Study product was supplied to the subjects monthly. At day 30, 60, and 90/Exit visits, assessments included anthropometrics, functional and nutritional status, study product intake, and quality of life (EQ-5D and SF-36). Additional contact via home visit or telephone was performed weekly to maintain study interest and study product intake.

### Treatment Compliance

Product compliance during inpatient hospitalization was closely monitored daily by study staff. Compliance during the post-discharge phase was assessed on monthly follow-up visits and at the final 90 day post-discharge study visit/Exit by evaluation of product diaries and return of unused product. Telephone or home visits were conducted weekly to encourage compliance to product and dietary intake or problem-solve any study issues.

### Discussion and Justification of Study Design

Choice of Study Design and Control Groups: A parallel design was chosen because a cross over design was inappropriate for this study. Subjects with heart failure, acute myocardial infarction, pneumonia, or chronic obstructive pulmonary disease are hospitalized for a short duration, i.e. approximately 6 days on average. It is unlikely that subjects will return to their original baseline following a washout period due to bed rest related loss of lean mass within such a short period.

### Measurement of Results

Efficacy Variable(s) include Quality of Life (EQ-5D and SF-36) at discharge and 30, 60, and 90 days post-discharge.

Supportive Variable(s) include: (1) Study product intake; and (2) Change of nutritional status (from Subjective Global Assessment) at hospital discharge, 30, 60, and 90 days post-discharge.

Demographic, Anthropometric, and Baseline Variables include: (1) Demographics: age, gender, ethnicity, race, marital status, primary diagnosis, household size [number of adults and children living in the household], household income, employment status [occupation if employed], health insurance type, education, and zip code; (2) Anthropometrics: height at screening, weight, and body mass index; and (3) Baseline Variables: distribution of nutritional status (Subjective Global Assessment).

The Subjective Global Assessment (SGA) was performed at screening and used for randomization. The SGA is a widely used and practical assessment tool of malnutrition, recommended by the American Society of Parenteral and Enteral Nutrition, providing a classification of malnutrition based on both medical history and clinician observations. The SGA is simple to administer and is widely used in research settings to document and classify patients into one of three categories of nutrition status: A=well-nourished; B=mildly to moderately malnourished; and C=severely malnourished.

The quality of life was assessed by two measures, the EQ-5D Instrument (“EQ-D5”) and the Short Form Health Survey (“SF-36”).

The EQ-5D is a generic, single index measure of health status developed by the EuroQol Group, a collaborative research network representing experts from countries around the world. Average scores, standardized by age/sex, for both the weighted index and the self-assessed visual analog scale (VAS), are available for the general population and for key population subgroups. EQ-5D is self-administered, reliable, and valid and is available in more than 20 languages. This quality of life measure has been used in many clinical trials (Kind 1998, 2002). EQ-5D has five questions based on the areas of mobility, self care, usual activities, pain/discomfort, and anxiety/depression, and was used to measure quality of life during the post-discharge phase.

The SF-36 Health Survey is a survey of patient health maintained and updated by QualityMetric Incorporated. The original SF-36 came out from the Medical Outcome Study (MOS) developed by the RAND Corporation. The SF-36 is used to conduct surveys of general and specific populations, comparing the relative burden of diverse diseases, and differentiating the health benefits produced by a wide range of treatments. The SF-36 has 36 questions that yield an eight-scale profile of functional health and wellbeing, as well as two summary measures of overall physical health and mental health. Each of the eight scales in the profile is a “domain,” evaluating physical functioning, physical roles, bodily pain, general health, vitality, social functioning, emotional roles, and mental health. The first four domains primarily contribute to the “physical health” component summary measurement, and the last four domains primarily contribute to the “mental health” component summary measurement.

### Statistical and Analytical Plans

This was a randomized, double-blinded, controlled, two-group, parallel design, multicenter intervention trial to evaluate the effects of consuming a high-calorie complete and balanced oral nutritional supplement providing additional protein and calcium HMB on improvements in quality of life in older hospitalized adults during and after hospitalization.

### Results: EQ-5D

When measured for all patients in the study population, there was no significant difference between the control group and experimental group for the EQ-5D index scores and health state scores. However, subgroups of the study population showed trends of improved EQ-5D index scores. Specifically, female patients in the experimental group (i.e., those administered HMB) showed a trend of higher EQ-5D index scores when compared with the female patients in the control group, with the experimental group showing an average 13.7% improvement (p=0.076). Also, a trend of improved EQ-5D index scores was observed for severely malnourished patients (i.e., SGA status C) in the experimental group, when compared to severely malnourished patients in the control group, with the experimental group showing an average 22.5% improvement (p=0.097).

### Results: SF-36

When measured for all patients in the study population, there was no significant difference between the control group and experimental group for the SF-36 physical health component score or mental health component score. However, subgroups of the study population showed trends of improved mental health component scores. Specifically, a trend of improved mental health component scores was observed for severely malnourished patients (i.e., SGA status C) in the experimental group, when compared to severely malnourished patients in the control group, with the experimental group showing an average 26.6% improvement (p=0.081).

When evaluating the SF-36 domain scores, certain subgroups of the study population also showed improved quality of life. Within the “General Health” domain, patients hospitalized for heart failure in the experimental group reported improved general health when compared with patients hospitalized for heart failure in the control group, with the experimental group showing an average 12.2% improvement (p=0.037). Also within the “General Health” domain, severely malnourished patients (i.e., SGA status C) in the experimental group showed a trend of improved general health when compared to the severely malnourished patients in the control group, with the experimental group showing an average 12.4% improvement (p=0.096). Similarly, within the “Mental Health” domain, severely malnourished patients in the experimental group reported improved mental health when compared to the severely malnourished patients in the control group, with the experimental group showing an average 32.4% improvement (p=0.021).

These study results suggest that administering a nutritional composition comprising HMB to adult or elderly patients during and after hospitalization may improve the quality of life for these patients. Improvements of greater than 10%, including from about 10% to about 35%, including from about 12% to about 32%, in both general and mental health were observed. The improvements were most striking in patients that were severely malnourished (i.e., SGA status C) upon admission to the hospital, but other subgroups, including females and patients hospitalized for heart failure, also recorded trends for improved quality of life in areas of general and mental health.

While the present application has been illustrated by the description of embodiments thereof, and while the embodiments have been described in considerable detail, it is not the intention of the applicant to restrict or in any way limit the scope of the appended claims to such detail. Additional advantages and modifications will readily appear to those skilled in the art. Therefore, the application, in its broader aspects, is not limited to the specific details, the representative compositions, formulations and methods, and illustrative examples shown and described. Accordingly, departures may be made from such details without departing from the spirit or scope of the applicant's general disclosure herein.

What is claimed is:

1. A method of improving quality of life in an adult patient subsequent to a hospitalization of the patient, comprising administering an effective amount of β-hydroxy-β-methylbutyrate to the patient after the hospitalization.

2. The method of claim 1, wherein the β-hydroxy-β-methylbutyrate is administered orally.

3. The method of claim 2, wherein the β-hydroxy-β-methylbutyrate is administered as part of a nutritional composition.

4. The method of claim 3, wherein the nutritional composition further comprises at least one source of protein, at least one source of carbohydrate, and at least one source of fat.

5. The method of claim 3, wherein the nutritional composition is in the form of a liquid, a powder suitable for reconstitution to a liquid, or a bar.

6. The method of claim 3, wherein the nutritional composition comprises about 350-500 kilocalories per serving and at least about 10 grams of protein per serving.

7. The method of claim 1, wherein the β-hydroxy-β-methylbutyrate comprises calcium β-hydroxy-β-methylbutyrate.

8. The method of claim 1, wherein the effective amount of β-hydroxy-β-methylbutyrate is from about 0.1 g/day to about 10 g/day.

9. The method of claim 1, wherein the β-hydroxy-β-methylbutyrate is administered at least once per day.

10. The method of claim 1, wherein the β-hydroxy-β-methylbutyrate is further administered to the patient during at least a portion of the hospitalization.

11. The method of claim 1, wherein the β-hydroxy-β-methylbutyrate is administered to the patient up to about 1 year after the hospitalization has ended.

12. The method of claim 1, wherein the patient is at least 65 years old.

13. The method of claim 1, wherein the patient was malnourished immediately prior to administration of the β-hydroxy-β-methylbutyrate.

14. The method of claim 1, wherein the improved quality of life comprises improved general health.

15. The method of claim 1, wherein the improved quality of life comprises improved mental health.

16. A method of improving quality of life in an elderly patient subsequent to a hospitalization of the patient, the method comprising administering from about 1 g/day to about 10 g/day of β-hydroxy-β-methylbutyrate in a nutritional composition to the elderly patient, wherein the administration occurs at least once per day up to about one year after the hospitalization has ended.

17. The method of claim 16, wherein the β-hydroxy-β-methylbutyrate is also administered to the elderly patient during the hospitalization.

18. The method of claim 16, wherein the nutritional composition is in the form of a liquid, a powder suitable for reconstitution to a liquid, or a bar.

19. The method of claim 16, wherein the nutritional composition comprises about 350-500 kilocalories per serving and at least about 10 grams of protein per serving.

20. The method of claim 16, wherein the elderly patient was malnourished immediately prior to administration of the β-hydroxy-β-methylbutyrate.

21. The method of claim 16, wherein the improved quality of life comprises improved general health.

22. The method of claim 16, wherein the improved quality of life comprises improved mental health.
